(19) United States
(12) Patent Application Publication
(10) Pub. No.: US 2006/0153778 A1
(43) Pub. Date: Jul. 13, 2006

(54) Methods and compositions for minimizing accrual of inhalable insulin in the lungs

(75) Inventors: Cohava Gelber, Manassas, VA (US); Anders Hasager Boss, Princeton, NJ (US)

Correspondence Address: PRESTON GATES & ELLIS LLP, 1900 MAIN STREET, SUITE 600, IRVINE, CA, 92614-7319, US

(73) Assignee: MannKind Corporation, Valencia, CA (US)

(21) Appl. No.: 11/330,427

(22) Filed: Jan. 10, 2006

Related U.S. Application Data
(60) Provisional application No. 60/643,054, filed on Jan. 10, 2005.

(51) Int. Cl. A61K 38/28; A61K 9/14; A61K 31/495; A61L 9/04

(52) U.S. Cl. 424/46; 514/3; 514/255.02

(57) ABSTRACT

Inhalable insulin compositions are provided that rapidly clear from the lungs of patients. Additionally, methods of minimizing insulin accrual after administration of an inhaled insulin composition are disclosed.

### CROSS REFERENCE TO RELATED APPLICATIONS

This application claims priority under 35 U.S.C. §119(e) to U.S. Provisional Application Ser. No. 60/643,054 filed Jan. 10, 2004.

### FIELD OF THE INVENTION

The present invention is related to methods and compositions for the delivery of inhalable protein drugs, such as insulin, to patients in need thereof. More specifically the present invention provides methods and compositions for delivery of inhalable insulin compositions to a patient's lungs.

### BACKGROUND OF THE INVENTION

In a normal person, the β-cells of the pancreatic islets of Langerhans produce insulin, required by the body for glucose metabolism, in response to an increase in blood glucose concentration. The insulin metabolizes incoming glucose and temporarily stops the liver's conversion of glycogen and lipids to glucose, thereby allowing the body to support metabolic activity between meals. The Type I diabetic, however, has a reduced ability or absolute inability to produce insulin due to β-cell destruction and needs to replace the insulin via daily injections or an insulin pump. More common than Type I diabetes, though, is Type II diabetes, which is characterized by insulin resistance and increasingly impaired pancreatic β-cell function. Type II diabetics may still produce insulin, but they may also require insulin replacement therapy.

Type II diabetics typically exhibit a delayed response to increases in blood glucose levels. While normal persons usually release insulin within 2-3 minutes following the consumption of food, Type II diabetics may not secrete endogenous insulin for several hours after consumption. As a result, endogenous glucose production continues after consumption (Pfeiffer, Am. J. Med., 70:579-88 (1981)), and the patient experiences hyperglycemia due to elevated blood glucose levels.

Loss of glucose-induced insulin secretion is one of the earliest disturbances of β-cell function (Cerasi et al., Diabetes, 21:224-34 (1972); Polonsky et al., N. Engl. J. Med., 318:1231-39 (1988)), but the causes and degree of β-cell dysfunction are unknown in most cases. While genetic factors play an important role, (Leahy, Curr. Opin. Endocrinol. Diabetes, 2:300-06 (1995)), some insulin secretory disturbances seem to be acquired and may be at least partially reversible through optimal glucose control. Optimal glucose control via insulin therapy after a meal can lead to a significant improvement in natural glucose-induced insulin release by requiring both normal tissue responsiveness to administered insulin and an abrupt increase in serum insulin concentrations. Therefore, the challenge presented in the treatment of early-stage Type II diabetics, those who do not have excessive loss of β-cell function, is to restore the release of insulin following meals.

Most early-stage Type II diabetics currently are treated with oral agents, but with little success. Subcutaneous injections of insulin are also rarely effective in providing insulin to Type II diabetics and may actually worsen insulin action because of delayed, variable, and shallow onset of action. It has been shown, however, that if insulin is administered intravenously with a meal, early stage Type II diabetics experience the shutdown of hepatic glucogenesis and exhibit increased physiological glucose control. In addition, their free fatty acids levels fall at a faster rate than without insulin therapy. While possibly effective in treating Type II diabetes, intravenous administration of insulin is not a reasonable solution, as it is not safe or feasible for patients to intravenously administer insulin at every meal.

Insulin, a polypeptide with a nominal molecular weight of 6,000 Daltons, traditionally has been produced by processing pig and cow pancreases to isolate the natural product. More recently, however, recombinant technology has been used to produce human insulin in vitro. Natural and recombinant human insulin in aqueous solution is in a hexameric configuration, that is, six molecules of recombinant insulin are noncovalently associated in a hexameric complex when dissolved in water in the presence of zinc ions. Hexameric insulin, however, is not rapidly absorbed. In order for recombinant human insulin to be absorbed into a patient's circulation, the hexameric form must first disassociate into dimeric and/or monomeric forms before the material can move into the blood stream. The delay in absorption requires that the recombinant human insulin be administered approximately one-half hour prior to meal time in order to produce therapeutic insulin blood levels, which can be burdensome to patients who are required to accurately anticipate the times they will be eating. To overcome this delay, analogs of recombinant human insulin, such as HUMALOG® (HUMALOG® is a registered trademark of Eli Lilly and Company), have been developed, which rapidly disassociate into a virtually entirely monomeric form following subcutaneous administration. Clinical studies have demonstrated that HUMALOG® is absorbed quantitatively faster than recombinant human insulin after subcutaneous administration. See, for example, U.S. Pat. No. 5,547,929 to Anderson Jr., et al.

In an effort to avoid the disadvantages associated with delivery by injection and to speed absorption, administration of monomeric analogs of insulin via the pulmonary route has been developed. For example, U.S. Pat. No. 5,888,477 to Gonda et al. discloses having a patient inhale an aerosolized formulation of monomeric insulin to deposit particles of insulin on the patient's lung tissue. However, the monomeric formulation is unstable and rapidly loses activity, while the rate of uptake remains unaltered.

While it would be desirable to produce rapidly absorbable insulin derived from natural sources, transformation of the hexameric form into the monomeric form, such as by removing the zinc from the complex, yields an insulin that is unstable and has an undesirably short shelf life. It therefore would be desirable to provide monomeric forms of insulin, which maintains its stability in the absence of zinc. It also would be advantageous to provide diabetic patients with monomeric insulin compositions that are suitable for pulmonary administration, provide rapid absorption, and which can be produced in ready-to-use formulations that have a commercially useful shelf-life, provide physiologic insulin levels and do not accumulate in the patient's lungs.

These problems with impurities, metal ions that affect stability or bioavailability, occur with many other proteins and peptides.

U.S. Pat. No. 6,071,497 to Steiner, et al. discloses microparticle drug delivery systems in which the drug is associated in diketopiperazine microparticles which are stable at a pH of 6.4 or less and unstable at pH of greater than 6.4, or which are stable at both acidic and basic pH, but which are unstable at pH between about 6.4 and 8. The patent does not describe monomeric insulin compositions that are suitable for pulmonary administration, provide rapid absorption, and which can be produced in ready-to-use formulations that have a commercially useful shelf-life.

One fear related to the development of pulmonary drug delivery is that lung function will be adversely affected. Rapid transit through the lung is seen as one way to minimize the likelihood of such an outcome. Thus, one of the goals of inhalation drug delivery is the rapid absorption of the drug from the lung tissue into the blood stream. Inhalation formulations of drugs, when inhaled, are generally absorbed through the epithelial cells of the alveolar region into the blood circulation. However, these drugs should be absorbed rapidly into the blood circulation and not left in contact with lung alveolar tissues.

It would therefore be advantageous to develop alternative insulin delivery compositions for Type II diabetics that provide more rapid elevation of insulin blood levels and are easily administered to ensure patient compliance and do not accumulate in the patient's lung tissue.

### SUMMARY OF THE INVENTION

Methods and compositions are provided for minimizing the accrual of inhaled insulin in the lungs of a patient after administration of an inhaled insulin composition.

In one embodiment of the present invention, a method is provided for minimizing insulin accrual in the lungs of a patient comprising providing an inhalable insulin composition to the patient in need thereof; administering the inhalable insulin composition to the patient's lungs; wherein the administering step is performed via inhalation; and wherein the inhaled insulin is cleared from the patient's lungs in less than approximately six hours, alternatively in less than approximately three hours.

In another embodiment of the methods of the present invention, the inhalable insulin composition is a dry powder. In yet another embodiment of the methods of the present invention, the providing step includes providing insulin complexed with a diketopiperazine, such as fumaryl diketopiperazine.

In another embodiment of the method of the present invention, a patient's lung function is not depressed on extended use of the inhalable insulin composition, wherein the patient's lung function is not impaired relative to the same patient not receiving an inhaled insulin composition.

In one embodiment of the present invention, an inhalable insulin composition is provided comprising insulin complexed with a diketopiperazine wherein the insulin is cleared from a patient's lungs in less than approximately six hours, alternatively in less than approximately three hours. In another embodiment the inhalable insulin composition is a dry powder. In yet another embodiment, the providing step includes providing insulin complexed with a diketopiperazine, such as fumaryl diketopiperazine.

In another embodiment of the present invention, the inhalable insulin composition comprises monomeric or dimeric insulin.

In another embodiment of the present invention, a patient's lung function is not depressed on extended use of the inhalable insulin composition, wherein the patient's lung function is not impaired relative to the same patient not receiving an inhaled insulin composition.

In an embodiment of the present invention, a method of treating diabetes is provided comprising providing an inhalable insulin composition to a patient in need thereof wherein extended use of the inhalable insulin composition does not impair lung function.

In another embodiment of the present invention, an inhalable insulin composition useful for treating diabetes is provided comprising an insulin/diketopiperazine complex wherein the inhalable insulin composition does not impair lung function.

### BRIEF DESCRIPTION OF THE DRAWINGS

FIGS. 1A and 1B depict the insulin lung pharmacokinetic profile following inhalation of 3 Units Technosphere®/Insulin daily for one or three days according to the teaching of one embodiment of the present invention.

FIGS. 2A and 2B depict the insulin Cₘₐₓ in lung (FIG. 2A) and serum (FIG. 2B) following inhalation of 3 Units Technosphere®/Insulin daily for one or three days according to the teaching of one embodiment of the present invention.

FIGS. 3A and 3B depict the insulin AUC₍₀₋ₗₐₛₜ₎ in lung (FIG. 3A) and serum (FIG. 3B) following inhalation of 3 Units Technosphere®/Insulin daily for one or three days according to the teaching of one embodiment of the present invention.

FIGS. 4A and 4B depict the insulin half-life (t_{1/2}) in lung (FIG. 4A) and serum (FIG. 4B) following inhalation of 3 Units Technosphere®/Insulin daily for one or three days according to the teaching of one embodiment of the present invention.

FIG. 5 graphically depicts the total levels of fumaryl diketopiperazine (FDKP) and insulin in the lungs post inhalation according to the teachings of one embodiment of the present invention.

FIGS. 6A and 6B depict pulmonary function, expressed as forced expiratory volume in one second (FEV₁, FIG. 6A) and forced vital capacity (FVC, FIG. 6B) over time in a three month placebo-controlled clinical study with Technosphere®/Insulin according to the teachings of the present invention.

FIG. 7 depicts changes in DLco from baseline to final treatment visit by final TI dosage group according to the teachings of one embodiment of the present invention.

FIG. 8 depicts changes in FEV₁ from baseline to final treatment visit by final TI dosage group according to the teachings of one embodiment of the present invention.

FIG. 9 depicts FEV₁ mean change from baseline from a study of patients receiving EXUBERA® (From: Advisory Committee Briefing Document: EXUBERA® (insulin [rDNA origin] powder for oral inhalation); Endocrinologic and Metabolic Drugs Advisory Committee Sep. 6, 2005).

### DETAILED DESCRIPTION OF THE INVENTION

The present invention is a method of minimizing accrual of insulin in the lungs of a patient after pulmonary administration of insulin compositions.

As used herein, the terms “complexation and “complexed” refer to a more intimate association than just entrapment or encapsulation would necessarily require, for example, binding based on charge or hydrophobicity.

As used herein, the term “Technosphere®/Insulin” refers to fumaryl diketopiperazine complexed with insulin. Technosphere® are microparticles (also referred to herein as microspheres) formed of diketopiperazine that self-assembles into an ordered lattice array at particular pHs, typically a low pH. They typically are produced to have a mean diameter between about 1 and about 5 μm.

As used herein, the term “extended use” refers to the regular administration of an insulin composition for at least three months.

Subcutaneous and intravenous insulin dosages are expressed in IU, which is defined by a standard biologic measurement. Amounts of insulin formulated with fumaryl diketopiperazine are also reported in IU as are measurements of insulin in the blood. Technosphere®/Insulin dosages are expressed in arbitrary units (U) which are numerically equivalent to the amount of insulin formulated in the dosage.

As used herein, the terms “active agent” and “drug” refer to any polymer or large organic molecules, most preferably peptides and proteins. Non-limiting examples include synthetic inorganic and organic compounds, proteins and peptides, polysaccharides and other sugars, lipids, and nucleic acid sequences having therapeutic, prophylactic or diagnostic activities. Proteins are defined as consisting of 100 amino acid residues or more; peptide are less than 100 amino acid residues. Unless otherwise stated, the term protein refers to both proteins and peptides. The active agents can have a variety of biological activities, including, but not limited to, vasoactive agents, neuroactive agents, hormones, anticoagulants, immunomodulating agents, cytotoxic agents, antibiotics, antivirals, antisense, antigens, and antibodies. In some instances, the proteins may be antibodies or antigens which otherwise would have to be administered by injection to elicit an appropriate response. Representative polymers include, but are not limited to, proteins, peptides, polysaccharides, nucleic acid molecule, and combinations thereof.

It was discovered that hexameric insulin can be delivered to the lung in a fumaryl diketopiperazine formulation, reaching peak blood concentrations within 3-10 minutes. In contrast, insulin administered by the pulmonary route without fumaryl diketopiperazine typically takes between 25-60 minutes to reach peak blood concentrations, while hexameric insulin takes 30-90 minutes to reach peak blood level when administered by subcutaneous injection. This observation has been successfully replicated several times and in several species, including humans.

Removing zinc from insulin typically produces unstable monomeric insulin with an undesirably short shelf life. Formulations of insulin complexed with fumaryl diketopiperazine were found to be stable and have an acceptable shelf life. Measurement of the zinc levels demonstrated that the zinc had been largely removed during the complexation process, yielding monomeric insulin in a stable delivery formulation.

Complexation of FDKP can increase the pulmonary absorption of a number of other peptides, including salmon calcitonin, parathyroid hormone 1-34, octreotide, leuprolide and RSV peptide, providing peak blood concentrations within 3-10 minutes after pulmonary delivery.

A wide variety of active agents can be complexed for pulmonary delivery. It may or may not be a charged species. Examples of classes of active agents suitable for use in the compositions and methods described herein include therapeutic, prophylactic, and diagnostic agents, as well as dietary supplements, such as vitamins.

Other nucleic acid sequences that can be utilized include, but are not limited to, antisense molecules which bind to complementary DNA to inhibit transcription, ribozyme molecules, and external guide sequences used to target cleavage by RNAase P.

As used herein, vectors are agents that transport the gene into targeted cells and include a promoter yielding expression of the gene in the cells into which it is delivered. Promoters can be general promoters, yielding expression in a variety of mammalian cells, or cell specific, or even nuclear versus cytoplasmic specific. These are known to those skilled in the art and can be constructed using standard molecular biology protocols. Vectors increasing penetration, such as lipids, liposomes, lipid conjugate forming molecules, surfactants, and other membrane permeability enhancing agents are commercially available and can be delivered with the nucleic acid.

Diketopiperazines useful for complexation with active agents in the present compositions and methods are described, for example, in U.S. Pat. No. 6,071,497, which is incorporated herein in its entirety.

The diketopiperazines or their substitution analogs are rigid planar rings with at least six ring atoms containing heteroatoms and unbonded electron pairs. One or both of the nitrogens can be replaced with oxygen to create the substitution analogs diketomorpholine and diketodioxane, respectively. Although it is possible to replace a nitrogen with a sulfur atom, this does not yield a stable structure.

The general formula for diketopiperazine and its analogs is shown below.

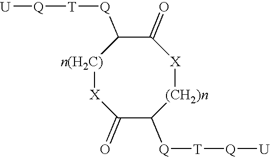

Wherein n is between 0 and 7, Q is, independently, a C₁₋₂₀ straight, branched or cyclic alkyl, aralkyl, alkaryl, alkenyl, alkynyl, heteroalkyl, heterocyclic, alkyl-heterocyclic, or heterocyclic-alkyl; T is C(O)O, —OC(O), —C(O)NH, —NH, —NQ, —OQO, —O, —NHC(O), —OP(O), —P(O)O, —OP(O)₂, —P(O)₂O, —OS(O)₂, or —S(O)₃; U is an acid group, such as a carboxylic acid, phosphoric acid, phosphonic acid and sulfonic acid, or a basic group, such as primary, secondary and tertiary amines, quaternary ammonium salts, guanidine, aniline, heterocyclic derivatives, such as pyridine and morpholine, or a zwitterionic C₁₋₂₀ chain containing at least one acidic group and at least one basic group, for example, those described above, wherein the side chains can be further functionalized with an alkene or alkyne group at any position, one or more of the carbons on the side chain can be replaced with an oxygen, for example, to provide short polyethylene glycol chains, one or more of the carbons can be functionalized with an acidic or basic group, as described above, and wherein the ring atoms X at positions 1 and 4 are either O or N.

As used herein, “side chains” are defined as Q-T-Q-U or Q-U, wherein Q, T, and U are defined above.

Examples of acidic side chains include, but are not limited, to cis and trans —CH═CH—CO₂H, —C(CH₃)═C(CH₃)—CO₂H, —(CH₂)₃—CO₂H, —CH₂CH(CH₃)—CO₂H, —CH(CH₂CO₂)—CH₂, -(tetrafluoro)benzoic acid, -benzoic acid and —CH(NHC(O)CF₃)—CH₂—CO₂H.

Examples of basic side chains include, but are not limited to, -aniline, -phenyl-C(NH)NH₂, -phenyl-C(NH)NH(alkyl), -phenyl-C(NH)N(alkyl)₂ and —(CH₂)₄NHC(O)CH(NH₂)CH(NH₂)CO₂H.

Examples of zwitterionic side chains include, but are not limited to, —CH(NH₂)—CH₂—CO₂ H and —NH(CH₂)₁₋₂₀CO₂H.

The term aralkyl refers to an aryl group with an alkyl substituent.

The term heterocyclic-alkyl refers to a heterocyclic group with an alkyl substituent.

The term alkaryl refers to an alkyl group that has an aryl substituent.

The term alkyl-heterocyclic refers to an alkyl group that has a heterocyclic substituent.

The term alkene, as referred to herein, and unless otherwise specified, refers to an alkene group of C₂ to C₁₀, and specifically includes vinyl and allyl.

The term alkyne, as referred to herein, and unless otherwise specified, refers to an alkyne group of C₂ to C₁₀.

As used herein, “diketopiperazines” includes diketopiperazines and derivatives and modifications thereof falling within the scope of the above-general formula.

Fumaryl diketopiperazine is most preferred for pulmonary applications.

Diketopiperazines can be formed by cyclodimerization of amino acid ester derivatives, as described by Katchalski, et al. (J. Amer. Chem. Soc. 68:879-80 (1946)), by cyclization of dipeptide ester derivatives, or by thermal dehydration of amino acid derivatives in high-boiling solvents, as described by Kopple, et al. (J. Org. Chem. 33(2):862-64 (1968)), the teachings of which are incorporated herein. 2,5-diketo-3,6-di(aminobutyl)piperazine (Katchalski et al. refer to this as lysine anhydride) was prepared via cyclodimerization of N-epsilon-P-L-lysine in molten phenol, similar to the Kopple method in J. Org. Chem., followed by removal of the blocking (P)-groups with 4.3 M HBr in acetic acid. This route is preferred because it uses a commercially available starting material, it involves reaction conditions that are reported to preserve stereochemistry of the starting materials in the product and all steps can be easily scaled up for manufacture.

Diketomorpholine and diketooxetane derivatives can be prepared by stepwise cyclization in a manner similar to that disclosed in Katchalski, et al.

Diketopiperazines can be radiolabelled. Means for attaching radiolabels are known to those skilled in the art. Radiolabelled diketopiperazines can be prepared, for example, by reacting tritium gas with those compounds listed above that contain a double or triple bond. A carbon-14 radiolabelled carbon can be incorporated into the side chain by using ¹⁴C labeled precursors which are readily available. These radiolabelled diketopiperazines can be detected in vivo after the resulting microparticles are administered to a subject.

Diketopiperazine derivatives are symmetrical when both side chains are identical. The side chains can contain acidic groups, basic groups, or combinations thereof.

One example of a symmetrical diketopiperazine derivative is 2,5-diketo-3,6-di(4-succinylaminobutyl)piperazine. 2,5-diketo-3,6-di(aminobutyl) piperazine is exhaustively succinylated with succinic anhydride in mildly alkaline aqueous solution to yield a product which is readily soluble in weakly alkaline aqueous solution, but which is quite insoluble in acidic aqueous solutions. When concentrated solutions of the compound in weakly alkaline media are rapidly acidified under appropriate conditions, the material separates from the solution as microparticles.

Other diketopiperazine derivatives can be obtained by replacing the succinyl group(s) in the above compound with glutaryl, maleyl or fumaryl groups.

One method for preparing unsymmetrical diketopiperazine derivatives is to protect functional groups on the side chain, selectively deprotect one of the side chains, react the deprotected functional group to form a first side chain, deprotect the second functional group, and react the deprotected functional group to form a second side chain.

Diketopiperazine derivatives with protected acidic side chains, such as cyclo-Lys(P)Lys(P), wherein P is a benzyloxycarbonyl group, or other protecting group known to those skilled in the art, can be selectively deprotected. The protecting groups can be selectively cleaved by using limiting reagents, such as HBr in the case of the benzyloxycarbonyl group, or fluoride ion in the case of silicon protecting groups, and by using controlled time intervals. In this manner, reaction mixtures which contain unprotected, monoprotected and di-protected diketopiperazine derivatives can be obtained. These compounds have different solubilities in various solvents and pH ranges, and can be separated by selective precipitation and removal. An appropriate solvent, for example, ether, can then be added to such reaction mixtures to precipitate all of these materials together. This can stop the deprotection reaction before completion by removing the diketopiperazines from the reactants used to deprotect the protecting groups. By stirring the mixed precipitate with water, both the partially and completely reacted species can be dissolved as salts in the aqueous medium. The unreacted starting material can be removed by centrifugation or filtration. By adjusting the pH of the aqueous solution to a weakly alkaline condition, the asymmetric monoprotected product containing a single protecting group precipitates from the solution, leaving the completely deprotected material in solution.

In the case of diketopiperazine derivatives with basic side chains, the basic groups can also be selectively deprotected. As described above, the deprotection step can be stopped before completion, for example, by adding a suitable solvent to the reaction. By carefully adjusting the solution pH, the deprotected derivative can be removed by filtration, leaving the partially and totally deprotected derivatives in solution. By adjusting the pH of the solution to a slightly acidic condition, the monoprotected derivative precipitates out of solution and can be isolated.

Zwitterionic diketopiperazine derivatives can also be selectively deprotected, as described above. In the last step, adjusting the pH to a slightly acidic condition precipitates the monoprotected compound with a free acidic group. Adjusting the pH to a slightly basic condition precipitates the monoprotected compound with a free basic group.

Limited removal of protecting groups by other mechanisms, including but not limited to cleaving protecting groups that are cleaved by hydrogenation by using a limited amount of hydrogen gas in the presence of palladium catalysts. The resulting product is also an asymmetric partially deprotected diketopiperazine derivative. These derivatives can be isolated essentially as described above.

The monoprotected diketopiperazine is reacted to produce a diketopiperazine with one sidechain and protecting group. Removal of protecting groups and coupling with other side chains yields unsymmetrically substituted diketopiperazines with a mix of acidic, basic, and zwitterionic sidechains.

Other materials that exhibit this response to pH can be obtained by functionalizing the amide ring nitrogens of the diketopiperazine ring.

Diketopiperazines can function as transport facilitators and are degradable and capable of forming hydrogen bonds with the target biological membrane in order to facilitate transport of the agent across the membrane. The transport facilitator can also be capable of forming hydrogen bonds with the active agent, if charged, in order to mask the charge and facilitate transport of the agent across the membrane.

The transport facilitator preferably is biodegradable and may provide linear, pulsed or bulk release of the active agent. The transport facilitator may be a natural or synthetic polymer and may be modified through substitutions or additions of chemical groups, including alkyly, alkylene, hydroxylations, oxidations, and other modifications routinely made by those skilled in the art.

Like most proteins and peptides, insulin is a charged molecule, which impedes its ability to cross charged biological membranes. It has been found that when insulin associates with fumaryl diketopiperazine, the passage of insulin across the membranes, such as mucosal membranes, and into the blood, is facilitated.

In one example, the active agent is associated within microparticles by dissolving a diketopiperazine with acidic side chains in bicarbonate or other basic solution, adding the active agent in solution or suspension, and then precipitating the microparticle by adding acid, such as 1 M citric acid.

In another example, the active agent is associated within microparticles by dissolving a diketopiperazine with basic side chains in an acidic solution, such as 1 M citric acid, adding the active agent in solution or suspension, and then precipitating the microparticle by adding bicarbonate or another basic solution.

In still another example, the active agent is associated within microparticles by dissolving a diketopiperazine with both acidic and basic side chains in an acidic or basic solution, adding the active agent in solution or suspension to be associated, then precipitating the microparticle by neutralizing the solution.

The microparticles can be stored in the dried state and suspended for administration to a patient. In a first example, the reconstituted microparticles maintain their stability in an acidic medium and dissociate as the medium approaches physiological pH in the range of between 6 and 14. In a second example, suspended microparticles maintain their stability in a basic medium and dissociate at a pH of between 0 and 6. In a third example, the reconstituted microparticles maintain their stability in an acidic or basic medium and dissociate as the medium approaches physiological pH in the range of pH between 6 and 8.

The impurities typically are removed when the microparticles are precipitated. However, impurities also can be removed by washing the particles to dissolve the impurities. A preferred wash solution is water or an aqueous buffer. Solvents other than water also can be used to wash the microspheres or precipitate the diketopiperazines, in order to remove impurities that are not water soluble. Any solvent in which neither the cargo nor the fumaryl diketopiperazine is soluble are suitable. Examples include acetic acid, ethanol, and toluene.

Microparticles of diketopiperazine can be prepared and provided in a suspension, typically an aqueous suspension, to which a solution of the active agent then is added. The suspension is then lyophilized or freeze-dried to yield diketopiperazine microparticles having a coating of active agent. In a preferred embodiment, the active agent is insulin in a hexameric form. Zinc ions can then be removed by washing the microparticles with an appropriate solvent.

The diketopiperazine microparticles have been found to efficiently bind insulin that is not bound to zinc, and after complexation, insulin is stabilized within an ordered lattice array of fumaryl diketopiperazine. In this state, in the sufficient absence of zinc ions, the insulin is predominately dimeric and monomeric, as opposed to the hexameric state. The insulin therefore more readily dissociates to its monomeric state, which is the state in which insulin exerts its biological activity.

The compositions of active agent described herein can be administered to patients in need of the active agent. The compositions preferably are administered in the form of microparticles, which can be in a dry powder form for pulmonary administration or suspended in an appropriate pharmaceutical carrier, such as saline.

The microparticles preferably are stored in dry or lyophilized form until immediately before administration. The microparticles then can be administered directly as a dry powder, such as by inhalation using, for example, dry powder inhalers known in the art. Alternatively, the microparticles can be suspended in a sufficient volume of pharmaceutical carrier, for example, as an aqueous solution for administration as an aerosol.

The microparticles also can be administered via oral, subcutaneous, and intraveneous routes.

The compositions can be administered to any targeted biological membrane, preferably a mucosal membrane of a patient, including a human suffering from Type II diabetes. The composition delivers insulin in biologically active form to the patient, which provides a spike of serum insulin concentration which simulates the normal response to eating.

In one embodiment, hexameric insulin is compleced with fumaryl diketopiperazine to form a solid precipitate of monomeric insulin in the fumaryl diketopiperazine, which then is washed with aqueous solution to remove the free zinc. This formulation demonstrates blood uptake following pulmonary administration at a rate 2.5 times the rate of insulin uptake following subcutaneous injection, with peak blood levels occurring at between 7.5 and 10 minutes after administration.

The range of loading of the drug to be delivered is typically between about 0.01% and 90%, depending on the form and size of the drug to be delivered and the target tissue. In one embodiment using diketopiperazines, the preferred range is from 0.1% to 50% loading by weight of drug. The appropriate dosage can be determined, for example, by the amount of incorporated/associated agent, the rate of its release from the microparticles, and, in a preferred embodiment, the patient's blood glucose level.

The compositions and methods described herein are further described by the following non-limiting examples.

### EXAMPLE 1

### Bioavailability of Insulin in Diketopiperazine Pulmonary Formulation

Five healthy male volunteers were evaluated for bioavailability of insulin after inhalation. The volunteers were in good health, as judged by physical examination, age: 18 to 40 years, body mass index: 18 to 26 kg/m², capability to reach peak inspiratory flow of ≧4 L/sec measured by a computer assisted spirometry and a FEV₁ (FEV₁=forced expiratory volume in one second) equal to or greater than 80% of predicted normal. Exclusion criteria were diabetes mellitus type 1 or 2, prevalence of human insulin antibodies, history of hypersensitivity to the study medication or to drugs with similar chemical structures, history or severe or multiple allergies, treatment with any other investigational drug in the last three months before study entry, progressive fatal disease, history of drug or alcohol abuse, current drug therapy with other drugs, history significant cardiovascular, respiratory, gastrointestinal, hepatic, renal, neurological, psychiatric and/or hematological disease, ongoing respiratory tract infection or subjects defined as being smokers with evidence or history of tobacco or nicotine use.

On the morning of the study days, the subjects came to the hospital (fasting, except for water, from midnight onward) at 7:30 a.m. The subjects were restricted from excessive physical activities and an intake of alcohol for 24 hours before each treatment day. They were randomly assigned to one of the three treatment arms. The subjects received a constant intravenous regular human insulin infusion, which was kept at 0.15 mU min-1 kg-1 so that serum insulin concentrations were established at 10-15 μU/ml during a period of two hours before time point 0. This low-dose infusion was continued throughout the test to suppress endogenous insulin secretion. Blood glucose was kept constant at a level of 90 mg/dL throughout the glucose clamp by a glucose controlled infusion system (BIOSTATOR™, Life Science Instruments, Miles Laboratories, Elkhart, Ind.). The glucose clamp algorithm was based on the actual measured blood glucose concentration and the grade of variability in the minutes before to calculate the glucose infusion rates for keeping the blood glucose concentration constant. The insulin application (5 IU intravenous (IV) or 10 IU subcutaneous (SC) injection or three deep breaths inhalation per capsule (2 capsules with 50 U each) of Technosphere®/Insulin applied with a commercial inhalation device (Boehringer Ingelheim)) had to be finished immediately before time point 0. The duration of the clamp experiment was 6 hours from time point 0. Glucose infusion rates, blood glucose, serum insulin and C-peptide were measured.

To determine bioefficacy, the areas under the curve of the glucose infusion rates were calculated for the first three hours (AUC₀₋₁₈₀) after the administration and for the overall observation period of six hours after the administration (AUC₀₋₃₆₀) and were correlated to the amount of insulin applied. To determine bioavailability, the areas under the curve of the insulin concentrations were calculated for the first three hours (AUC₀₋₁₈₀) after the administration and for the overall observation period of six hours after the administration (AUC₀₋₃₆₀) and correlated to the amount of insulin applied.

In this clamp study, inhalation of 100 U of Technosphere®/Insulin was well tolerated and was demonstrated to have a substantial blood glucose lowering effect with a relative bioavailability of 25.8% for the first three hours as calculated from the achieved serum insulin concentrations. Technosphere® are microparticles (also referred to herein as microspheres) formed of diketopiperazine that self-assembles into an ordered lattice array at particular pHs, typically a low pH. They typically are produced to have a mean diameter between about 1 and about 5 μm.

The pharmacokinetic results are illustrated in Table 1. Inhalation of 100 U of Technosphere®/Insulin revealed a peak of insulin concentration after 13 min (5 IU IV: 5 min, 10 IU SC: 121 min) and a return of the insulin levels to baseline after 180 min (IV: 60 min, SC: 360 min). Biological action as measured by glucose infusion rate peaked after 39 min (IV: 14 min, SC: 163 min) and lasted for more than 360 min (IV: 240 min, SC: >360 min). Absolute bioavailability (comparison to IV administration) was 14.6±5.1% for the first three hours and 15.5±5.6% for the first six hours. Relative bioavailability (comparison to SC administration) was 25.8±11.7% for the first three hours and 16.4±7.9% for the first six hours.

TABLE 1
Pharmacokinetic Parameters after Pulmonary
Administration of TI
Pharmacokinetic Parameters

| | | |
|---|---|---|
| Intravenous | | Subcutaneous |
| Administration | Inhaled | Administration |

Parameter Calculated on Glucose Infusion Rate

| | | | | | | |
|---|---|---|---|---|---|---|
| T50% * | 9 | min | 13 | min | 60 | min |
| Tmax | 14 | min | 39 | min | 163 | min |
| T-50% ** | 82 | min | 240 | min | 240 | min |
| T to baseline | 240 | min | >360 | min | >360 | min |

Parameter Calculated on Insulin Levels

| | | | | | | |
|---|---|---|---|---|---|---|
| T50% * | 2 | min | 2.5 | min | 27 | min |
| Tmax | 5 | min | 13 | min | 121 | min |
| T-50% ** | 6 | min | 35 | min | 250 | min |
| T to baseline | 60 | min | 180 | min | 360 | min |

* time from baseline to half-maximal values
** time from baseline to half-maximal after passing Tmax

Technosphere®/Insulin was shown to be safe in all patients. One patient was coughing during the inhalation without any further symptoms or signs of deterioration of the breathing system.

Inhalation of 100 U of Technosphere®/Insulin was well tolerated and was demonstrated to have a substantial blood glucose lowering effect with a relative bioavailability of 25.8% for the first three hours as calculated from the achieved serum insulin concentrations.

In this study, the inhalation of Technosphere®/Insulin was demonstrated in healthy human subjects to have a time-action profile with a rapid peak of insulin concentration (Tmax: 13 min) and rapid onset of action (Tmax: 39 min) and a sustained action over more than six hours. The total metabolic effect measured after inhalation of 100 U of Technosphere®/Insulin was larger than after subcutaneous injection of 10 IU of insulin. The relative bioefficacy of Technosphere®/Insulin was calculated to be 19.0%, while the relative bioavailability was determined to be 25.8% in the first three hours.

The data also show that inhalation of Technosphere®/Insulin resulted in a much more rapid onset of action than sc insulin injection that was close to the onset of action of IV insulin injection, while duration of action of Technosphere®/Insulin was comparable to that of SC insulin injection.

### EXAMPLE 2

### Lung and Serum Insulin Levels Following Administration of Technosphere®/Insulin

Lung and serum levels of insulin were determined after a single dose of Technosphere®/Insulin or after three daily doses of Technosphere®/Insulin.

Six female Sprague Dawley rats per group were treated with fumaryl diketopiperazine-insulin (Technosphere®/Insulin) 11.4% using a flow-past, nose-only inhalation exposure system with either a single dose or a single daily dose for three consecutive days. Approximately 3 Units of insulin was administered to each group via a flow-past, nose only inhalation chamber. Rats individual respiratory patterns were monitored, and the accumulated volume of inhalation was calculated for each animal. Administration was continued until the desired dose was achieved. Animals were evaluated after an air alone control and at 0, 45, 90 and 180 minutes and 6, 24 and 30 hours after Technosphere®/Insulin administrations. At each time point serum was obtained and the lungs removed to determine insulin levels.

TABLE 2
Insulin Pharmacokinetic Metrics from the Rat Lung and Serum

| | |
|---|---|
| Lung | Serum |

| Metric | Day 1 | Day 3 | Day 1 | Day 3 |
|---|---|---|---|---|
| Cₘₐₓ^{a} | 947 | 909 | 168 | 90.4 |
| tₘₐₓ (min) | 0 | 0 | 0 | 0 |
| AUCₗₐₛₜ^{b} | 67104 | 54720 | 13817 | 6901 |
| t_{1/2}^{c} (min) | 47.7 | 51.4 | 66.9 | 107.2^{d} |

^{a}lung units are mlU/rat lungs; serum units are μIU/mL
^{b}lung units are mlU*min/rat lung; serum units are μIU*min/mL
^{c}half-life is for initial (through 3 hours) clearance
^{d}value skewed during one of the two experiments

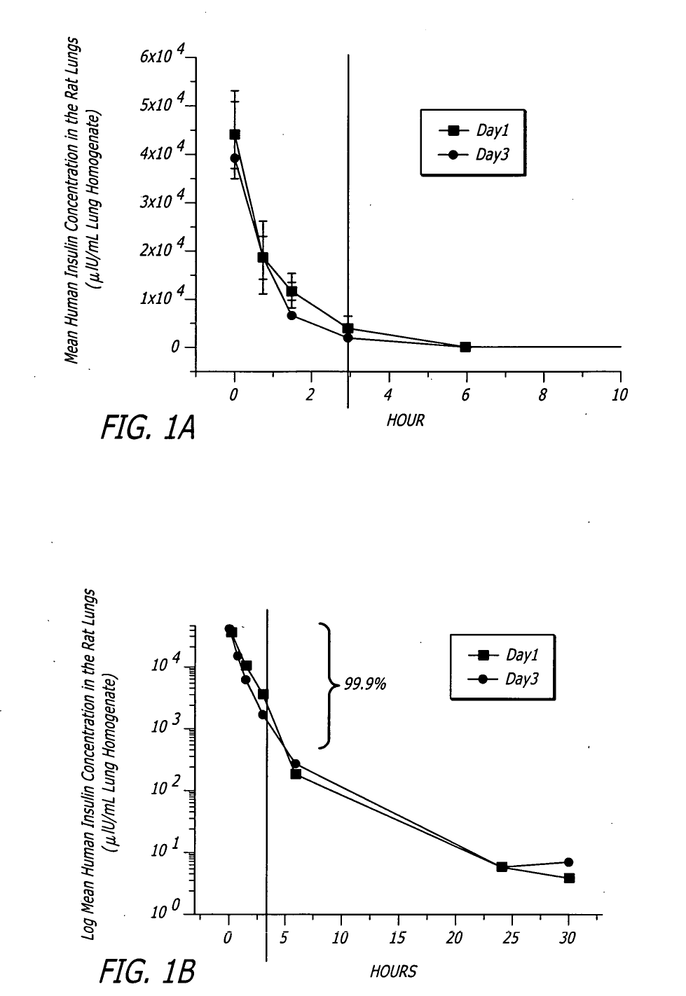

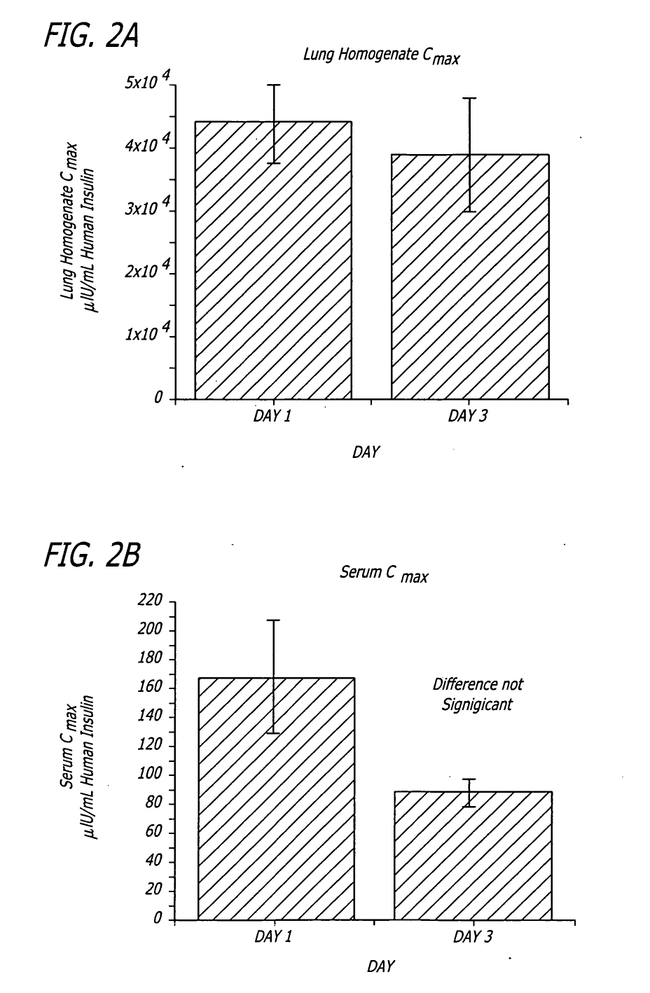

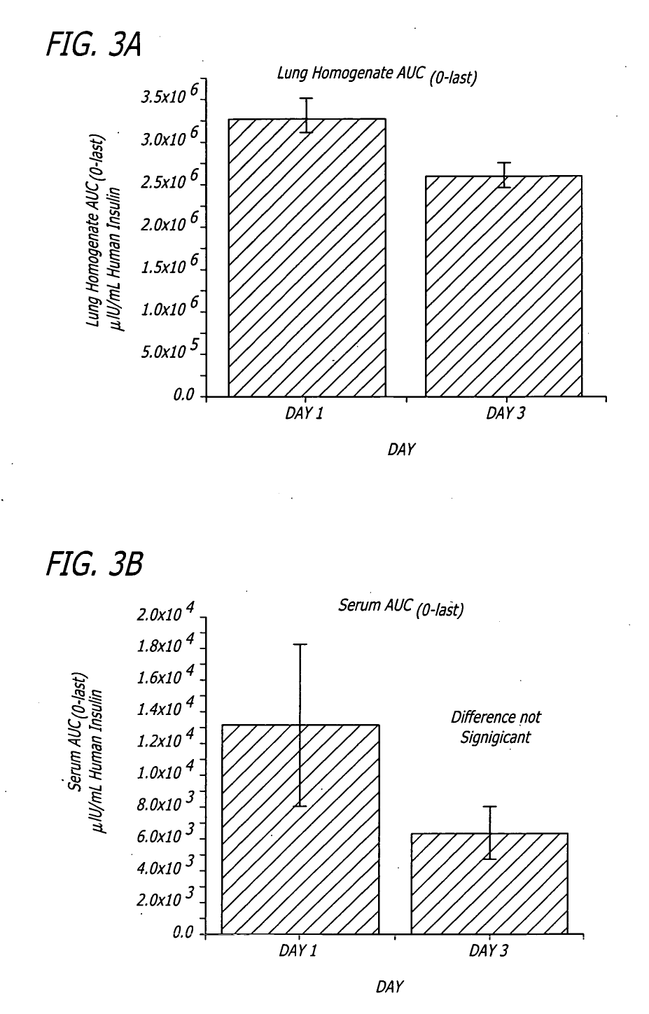

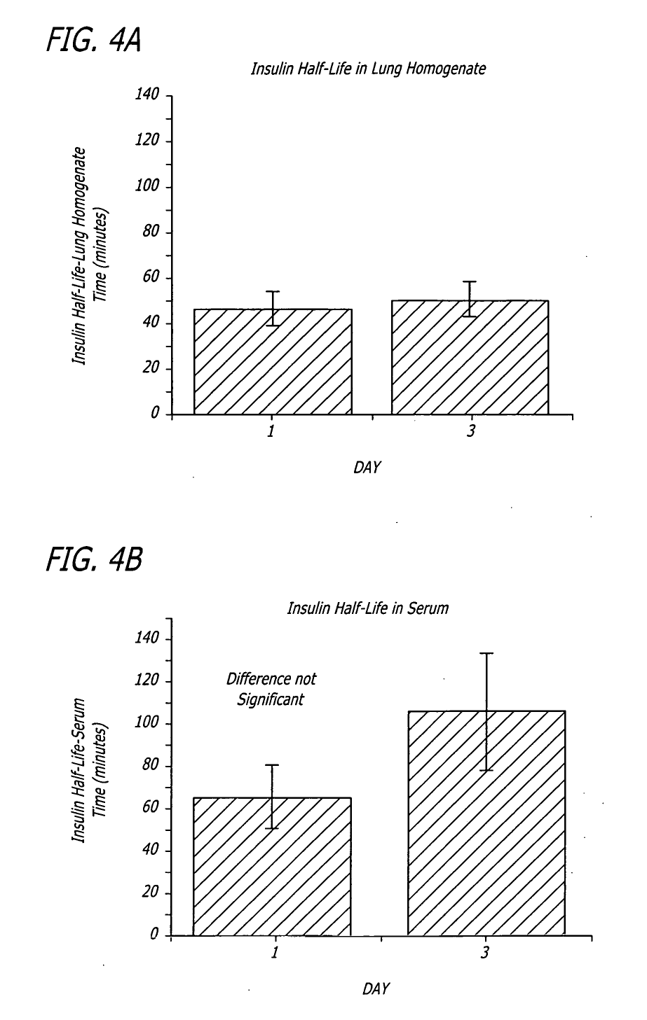

Lung exposure to insulin (mean Cₘₐₓ and AUCₗₐₛₜ) were comparable for both Day 1 and Day 3, with a rapid tₘₐₓ of time zero, i.e. immediately post dose (FIGS. 1, 2 and 3). The initial clearance is rapid, with a t_{1/2} of 45 minutes to 1 hour (FIG. 4). The serum however appeared to trend towards a lower mean Cₘₐₓ and AUCₗₐₛₜ on Day 3 (FIGS. 2 and 3). The serum t_{1/2} is slightly under 2 hours on day 3 (FIG. 4). There was a rapid, reproducible absorption and clearance of insulin from the lung tissues and systemic circulation, even following subsequent daily dosing, and there was no insulin accrual. Levels of Technosphere®/Insulin in the lungs began dropping within 45 minutes of inhalation with the majority of the insulin cleared within approximately 3 hours to approximately 6 hours.

### EXAMPLE 3

### Transit of Insulin and FDKP from the Lungs

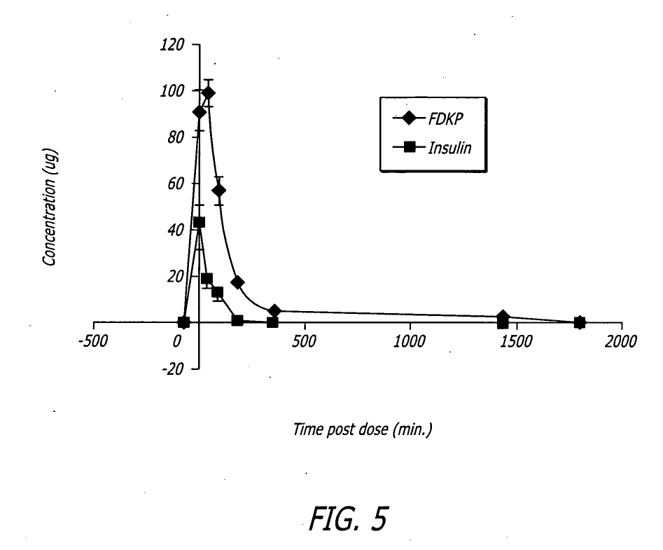

In an experiment essentially similar to Example 2, the transit of FDKP was followed in addition to insulin. As seen in FIG. 5, FDKP transited the lungs with kinetics similar to that of insulin. This demonstrated that both major components of Technosphere®/Insulin maintain a constant concentration ratio, and that neither is preferentially retained in the lungs.

### EXAMPLE 4

### Administration of Technosphere®/Insulin does not Cause a Decline in Measurements of Pulmonary Function

In a randomized, prospective double blind, placebo controlled study of the forced titration of prandial Technosphere®/Insulin in patients with type 2 diabetes mellitus subjects received inhaled Technosphere®/Insulin (TI), dosed prandially, in addition to basal administration of SC insulin glargine (Lantus®; a form of long acting insulin), 227 patients were studied over 18 weeks. During the initial 4 weeks, patients were followed on their existing therapy and then removed from all oral anti-hyperglycemic therapy and were placed on fixed doses of SC insulin glargine taken once daily, in a dose sufficient to replicate their documented pre-manipulation fasting plasma glucose levels and stabilized at this dose. The patients were then randomized to blinded doses of added inhaled placebo or blinded doses of inhaled TI containing 14, 28, 42 or 56 U of regular human insulin taken at the time of each main meal of the day in a forced titration scenario over 4 weeks. Specifically, the subjects, divided into five cohorts, initially received placebo (Technosphere® microparticles without any insulin) along with the sc long acting insulin. After a week one cohort continued to receive placebo and four cohorts were switched to a TI dose of 14 U of insulin. After another week three cohorts were switched to a TI dose of 28 U, and so on until a final cohort reached a TI dose of 56 U. All cohorts then continued on the same dose for the remaining eight weeks of the trial.

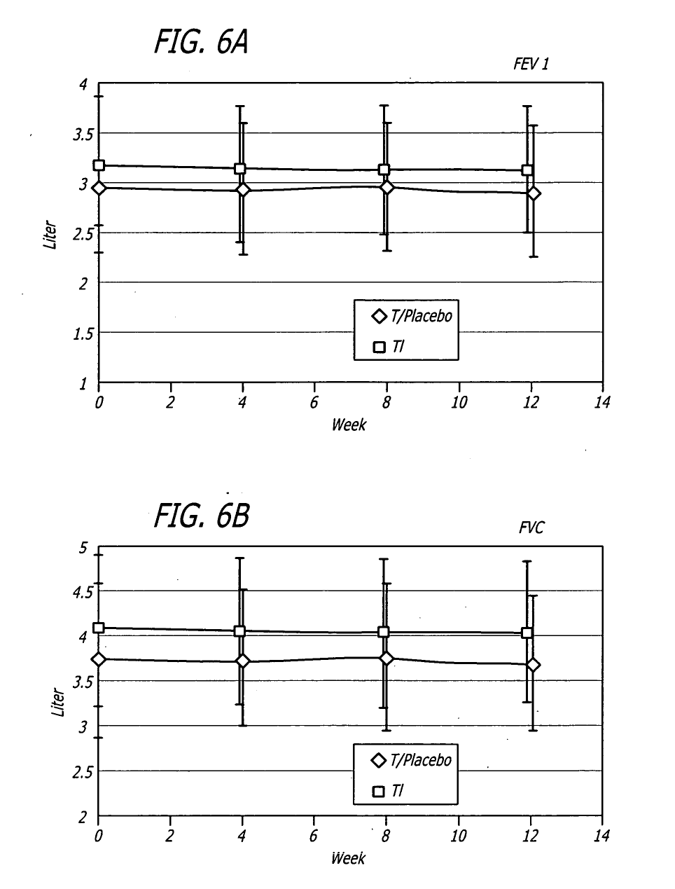

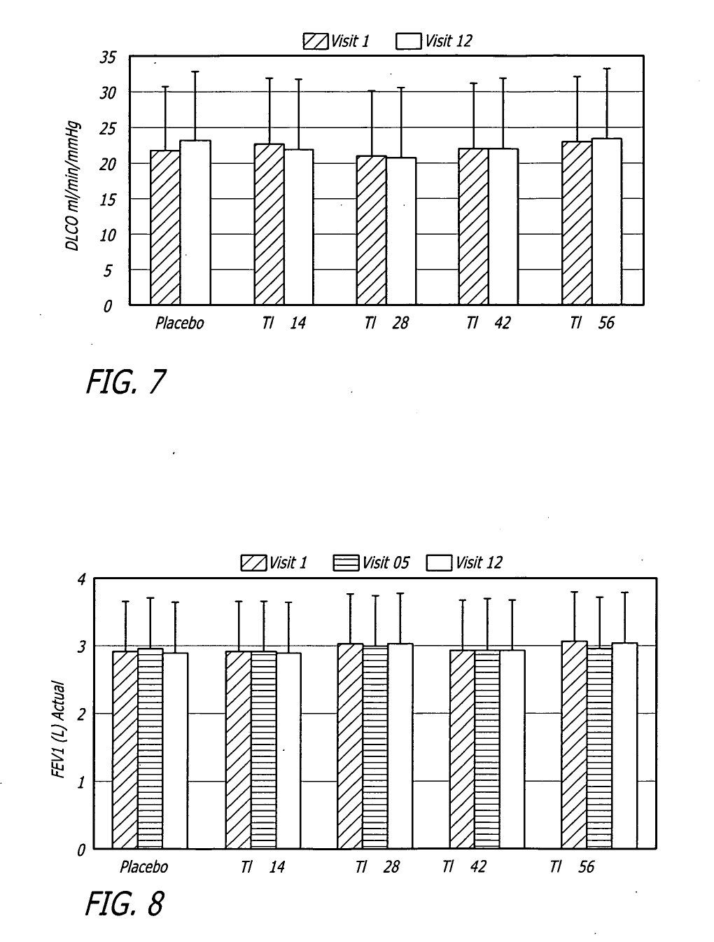

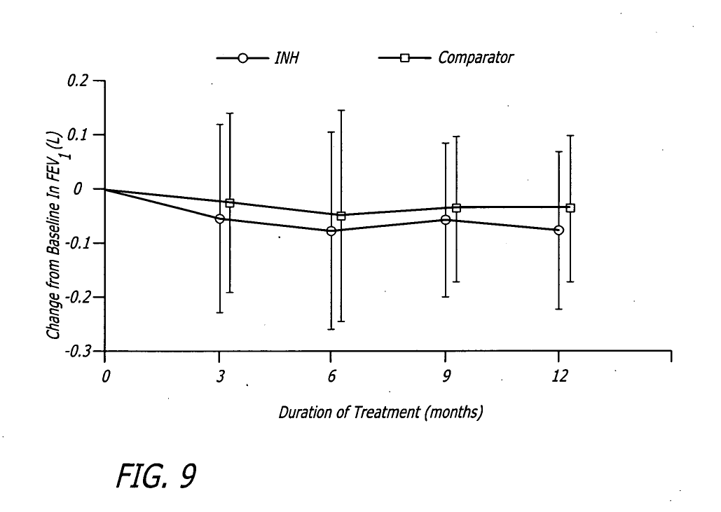

HbA1c levels and meal challenges (300 min) were evaluated at the initial visit, at the start of randomized treatment and at completion. Comparisons were made between treatment groups and the placebo group. Safety was assessed by the frequency of defined hypoglycemic episodes and by the measurement of serial pulmonary function tests including FEV₁ (forced expiratory volume in 1 second), and DL_{CO} (single breath carbon monoxide diffusion capacity). The addition of TI to insulin glargine produced a dose-dependent reduction in HbA1c levels. In patients treated for 8 weeks at 56 units, the mean reduction was 0.79% greater than that observed in the insulin glargine/placebo group (p=0.0002). TI also produced a dose-dependent reduction in post-prandial glucose excursions with a maximal excursion averaging only 34 mg/dL at 56 U (p<0.0001). There were no severe hypoglycemic episodes, and the frequency of mild/moderate hypoglycemic episodes was not increased above that in subjects on insulin glargine alone. No changes were observed from baseline or between dosage groups in weight or pulmonary function (FIGS. 6 and 7). Thus inhaled Technosphere®/Insulin was able to improve the glycemic control of patients with type 2 diabetes without increasing the risk of hypoglycemia.

The absence of change in pulmonary function with TI is in contrast with the reported observations with a pulmonary insulin product awaiting FDA approval (EXUBERA®). With that product by three months of use—the duration of the TI study above—there was a small but distinct drop in pulmonary function measured both as Dlco or FEV₁. After that time point the pulmonary function stabilized in relation to a comparator group not receiving pulmonary insulin, but remained depressed in comparison (see, for example, FIG. 8). Similar behavior was observed in multiple studies involving variously type 1 and type 2 diabetics and extending for as long as two years (Advisory Committee Briefing Document: EXUBERA® (insulin [rDNA origin] powder for oral inhalation); Endocrinologic and Metabolic Drugs Advisory Committee Sep. 6, 2005).

### EXAMPLE 5

### A Randomized, Double-blind, Placebo Controlled Study of the Efficacy and Safety of Inhaled Technosphere®/Insulin in Patients with Type 2 Diabetes

Technosphere® dry powder, pulmonary insulin delivered via the small MannKind™ inhaler has a bioavailability that mimics normal, meal-related, first- or early-phase insulin release. This multicenter, randomized, double-blind, placebo-controlled study was conducted in type 2 diabetes mellitus patients inadequately controlled on diet or oral agent therapy (HbA1c>6.5% to 10.5%). A total of 123 patients were enrolled and 119, the intention-to-treat population (ITT), were randomized in a 1:1 ratio to receive prandial inhaled Technosphere®/Insulin (TI) from unit dose cartridges containing between 6 to 48 units of human insulin (rDNA origin) or inhaled Technosphere®/placebo for 12 weeks. TI was inhaled at the time of the first mouthful of food at each main or substantive meal of the day, amounting to 3 or 4 administrations per day throughout the 12 week trial. Subjects continued whatever oral diabetes drugs they were using prior to entering the study. Differences in HbA1c from the first and final treatment visits, and between the first and two intermediate visits, were determined, as was the change in blood glucose, as AUC at various time points, and Cₘₐₓ and Tₘₐₓ, after a meal challenge.

Patients were given a standardized meal several times during the study and their blood glucose levels measured. The study drug was administered at the study site in conjunction with a standardized breakfast (Uncle Ben's Breakfast Bowl™) that was prepared at the site. Fasting plasma glucose was measured immediately before the meal. Spirometry was performed before the subject took the first dose of study drug. Subjects then inhaled the study drug and, within 60 seconds, performed a single spirometry test procedure. Within 90 seconds of the study drug inhalation, and after the spirometry test, the subject began eating the test meal. Once the meal was completed, the plasma glucose values and glucose meter readings were obtained at immediately before and at 30, 60 and 120 minutes after beginning the meal.

For patients receiving either TI or placebo, blood glucose rose after meal challenge, but significantly less for the TI group and returned to baseline sooner. Thus total glucose exposure and maximal glucose excursion were reduced. At a dose of 30 U the maximal glucose excursions for the TI patients were 50% of the level for the patients in the control group. Additionally, the average glucose excursion was about 28 mg/dL vs. 50 mg/dL when the TI patients entered the study. An excursion of only 28 mg/dL is within the range that is a goal of clinical treatment.

Glycosylated hemoglobin A1c (HbA1c) results were analyzed by a pre-determined statistical analysis plan for the Primary Efficacy Population (PEP, defined prior to un-blinding as those who adhered to study requirements including minimal dosing and no adjustments of concomitant diabetes drugs), for a PEP Sub-group A (those with baseline HbA1c of 6.6 to 7.9%). for a PEP Sub-group B (those with baseline HbA1c of 8.0 to 10.5%), as well as for the ITT. These results are summarized in Table 3. In this “individualized dose” study, the mean dose of TI used before each meal in the active treatment group was approximately 30 units, with 28 units used in PEP Sub-group A and 33.5 units used in PEP Sub-group B.

TABLE 3
HbA1c Pharmacokinetics

| | Technosphere ®/ | Technosphere ®/ |
|---|---|---|
| | Placebo | Insulin |
| PEP n = 90 | n = 42 | n = 48 |
| Mean HbA1c Baseline (%) | 7.75 | 7.74 |
| Mean Δ from baseline | −0.32 (p = 0.0028) | −0.76 (p < 0.0001) |
| Comparison to Placebo | | p = 0.0019 |
| PEP Sub-group B n = 35 | n = 18 | n = 17 |
| Mean HbA1c Baseline (%) | 8.52 | 8.72 |
| Mean Δ from baseline | −0.51 (p = 0.0094) | −1.37 (p < 0.0001) |
| Comparison to Placebo | | p = 0.0007 |
| PEP Sub-group A n = 55 | n = 24 | n = 31 |
| Mean HbA1c Baseline (%) | 7.16 | 7.19 |
| Mean Δ from baseline | −0.18 (p = 0.1292) | −0.43 (p = 0.0001) |
| Comparison to Placebo | | p < 0.05 |
| IIT (LOCF) n = 119 | n = 61 | n = 58 |
| Mean HbA1c Baseline (%) | 7.78 | 7.87 |
| Mean Δ from Baseline (%) | −0.31 (p = 0.0020) | −0.72 (p < 0.0001) |
| Comparison to Placebo | | p = 0.0016 |

No episodes of severe hypoglycemia occurred in the TI group. There was no statistically significant difference in the rate of hypoglycemic events between those subjects receiving placebo and those receiving TI. (Table 4).

TABLE 4
Incidence of Hypoglycemia after Pulmonary Administration of TI

| | Technosphere ®/Insulin | Technosphere ®/Placebo |
|---|---|---|
| Hypoglycemia | 42.6% | 35.5% |
| (% of patients) | | |
| Hypoglycemia | 0.16 | 0.20 |
| (events/week) | | |

Pulmonary function tests, including DLco (diffusing capacity of the lung for carbon monoxide) (Table 5), FEV1 (forced expiratory volume in one second), and total alveolar volume (forced vital capacity, FVC) showed no significant differences between patients on TI compared to their baseline values or compared to the results of those receiving placebo (FIG. 6).

TABLE 5
Pulmonary Function After Pulmonary Administration of TI

| DLco | Technosphere ®/Insulin | Technosphere ®/Placebo |
|---|---|---|
| 0 weeks | 24.9 ± 4.8 | 26.5 ± 5.6 |
| 12 weeks | 25.0 ± 4.5 | 25.7 ± 5.2 |

There was no evidence of induction of insulin antibodies with TI (Table 6) or of weight gain during the 12 week period of exposure.

TABLE 6
Incidence of Antibodies to Insulin after
Pulmonary Administration of TI

| | Technosphere ®/Insulin | Technosphere ®/Placebo |
|---|---|---|
| Negative at Visit 1/ | 38 | 34 |
| Negative at Visit 9 | | |
| Negative at Visit 1/ | 2 | 3 |
| Positive at Visit 9 | | |
| Positive at Visit 1/ | 8 | 10 |
| Positive at Visit 9 | | |
| Positive at Visit 1/ | 2 | 4 |
| Negative at Visit 9 | | |

In conclusion, this study has demonstrated that Technosphere® pulmonary insulin, in replication of the kinetics of the early phase of insulin release, when used in patients with inadequate glycemic control previously on only diet and exercise alone or on oral agent therapy, safely and significantly improved glycemic control with no significantly increased incidence of hypoglycemia, no induction of insulin antibodies, no tendency toward weight gain, and no evidence of overall impact on pulmonary function.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method for minimizing insulin accrual in the lungs of a patient after the administration of an inhaled insulin comprising:
providing said inhalable insulin composition to a patient in need thereof;
administering said inhalable insulin composition to said patient's lungs; wherein said administering is performed via inhalation; and
wherein said inhaled insulin is cleared from said patient's lungs in less than approximately six hours

2. The method according to claim 1 wherein said inhalable insulin composition is a dry powder.

3. The method according to claim 1 wherein said providing step includes providing insulin complexed with a diketopiperazine.

4. The method according to claim 3 wherein said diketopiperazine is fumaryl diketopiperazine.

5. The method according to claim 1 wherein said inhaled insulin is cleared from said patient's lungs in less than approximately three hours.

6. The method accord to claim 1 wherein a patient's lung function is not depressed on extended use of said inhaled insulin composition, wherein said patient's lung function is not impaired.

7. The method accord to claim 6 wherein a patient's lung function is not depressed on extended use of said inhaled insulin composition, wherein said patient's lung function is not impaired relative to the same patient not receiving an inhaled insulin composition.

8. An inhalable insulin composition comprising:
an insulin/diketopiperazine complex wherein said insulin is cleared from a patient's lungs in less than approximately six hours after inhalation.

9. The inhalable insulin composition of claim 8 wherein said insulin is cleared from said patient's lungs in less than approximately three hours.

10. The inhalable insulin composition of claim 8 wherein said inhalable insulin composition is a dry powder.

11. The inhalable insulin composition of claim 10 wherein said diketopiperazine is fumaryl diketopiperazine.

12. The inhalable insulin composition of claim 8 wherein said insulin is monomeric or dimeric.

13. The inhalable insulin composition of claim 8 wherein said diketopiperazine is cleared from a patient's lung in less than 6 hours.

14. The inhalable insulin composition of claim 8 wherein a patient's lung function is not depressed on extended use of said inhaled insulin composition, wherein said patient's lung function is not impaired.

15. The inhalable insulin composition of claim 14 wherein a patient's lung function is not depressed on extended use of said inhaled insulin composition, wherein said patient's lung function is not impaired relative to the same patient not receiving an inhaled insulin composition.

16. A method of treating diabetes comprising
providing an inhalable insulin composition to a patient in need thereof wherein extended use of said inhalable insulin composition does not impair lung function.

17. An inhalable insulin composition useful for treating diabetes comprising:
an insulin/diketopiperazine complex wherein said inhalable insulin composition does not impair lung function.
